(19) United States
(12) Patent Application Publication
(10) Pub. No.: US 2002/0002348 A1
(43) Pub. Date: Jan. 3, 2002

(54) Low profile metacarpal fracture brace

(76) Inventors: Chris E. Wiggins, Pascagoula, MS (US); Tom Young, Pascagoula, MS (US)

Correspondence Address: Roger A. Marrs, Suite 1120, 15233 Ventura Blvd., Sherman Oaks, CA, 91403, US

(21) Appl. No.: 09/886,857

(22) Filed: Jun. 21, 2001

Related U.S. Application Data
(60) Provisional application No. 60/214,366, filed on Jun. 28, 2000.

(51) Int. Cl. A61F 5/00

(52) U.S. Cl. 602/21

(57) ABSTRACT

A fracture brace having a U-shaped member with an inner, soft-cushioned layer carried on an outer rigid layer defining an open inner cavity for receiving and supporting a user's hand, adjacent to the metacarpal grouping of hand bones. The member is held in position by a strapping system which includes a lower wrist strap, a middle palm strap and an upper palm strap immediately below the fingers. One end of each strap is fixed to the backside of the member and securement to the member includes a loop associated with each strap. Each strap further includes a two-component hook and pile fastener. The pile component of the fastener is carried along the backside of each strap and each strap terminates with a free-end carrying the hook component of the fastener at the tip of the inside surface of each strap. The free end of each strap is adapted to be trained through associated loops of the strapping assembly. A spacer or cushion element is carried on the mid-strap and serves to cushion or separate the thumb from the index finger when the brace is worn on a hand. The inventive brace extends from the base of the hypothenar eminence to beyond the fifth metacarpal-phalangeal joint to support the base of the proximal phalanx of the fifth finger.

### BACKGROUND OF THE INVENTION

1. Field of the Invention

The present invention relates to the field of medical braces for fractures involving bones of the hand and more particularly to a brace for immobilizing fractures of metacarpal bones while allowing some freedom of finger movement.

2. Brief Description of the Prior Art

Conventionally, when fractures of hand bones occur, physicians generally employ immobilization using traditional methods such as employing casts or splints. This is particularly traditional for treating 4th and 5th metacarpal fractures of hand bones. Such traditional methods greatly restrict movement of the fingers and thereby increase the likelihood of joint stiffness. Furthermore, casts or splints greatly restrict active lifestyles and are not suited to be worn in sporting or industrial settings. Also, conventional casts or splints greatly reduce the ability of a patient to bathe such the cast or splints are not readily removable.

Therefore, a long-standing need has existed to provide a fracture brace which is easier for a physician to apply than other traditional methods, particularly for treating fourth and fifth metacarpal fractures. The fracture brace should be designed to immobilize fractures of the metacarpals and should have a low profile so that the brace may be employed in active lifestyles and is suitably removable for patient bathing.

### SUMMARY OF THE INVENTION

Accordingly, the above problems and difficulties are avoided by the present invention which provides a fracture brace having a U-shaped member with an inner, soft-cushioned layer carried on an outer rigid layer defining an open inner cavity for receiving and supporting the underside of the user's hand, adjacent to the metacarpal grouping of hand bones. The member is held in position by a strapping system which includes a lower wrist strap, a middle palm strap and an upper palm strap immediately below the fingers. One end of each strap is fixed to the backside of the member and the fixture to the member includes a loop associated with each strap. Each strap further includes a two-component hook and pile fastener or connection. The pile component of the fastener is carried along the backside of each strap and each strap terminates with a free-end carrying the second or hook component of the fastener at the tip of the inside surface of each strap. The free end of each strap is adapted to be trained through associated loops of the strapping assembly. A spacer or cushion element is carried on the mid-strap and serves to cushion or separate the thumb from the index finger when the brace is worn on a hand. Therefore, it can be seen that the inventive brace extends from the base of the hypothenar eminence to beyond the fifth metacarpal-phalangeal joint to support the base of the proximal phalanx of the fifth finger. The brace member is maintained in position by the strap assembly which runs transversely around the hand.

Therefore, it is among the primary objects of the present invention to provide a low-profile metacarpal fracture brace which will readily immobilize fractures of the fourth metacarpal and the fifth metacarpal in particular, while permitting limited movement of the fingers.

Another object of the present invention is to provide a fracture brace for protecting the metacarpals of the hand which is removably held in position by a strap assembly and which can readily be removed by the person wearing the brace for bathing purposes.

Yet another object of the present invention is to provide a low profile metacarpal fracture brace which allows more freedom of motion of the fingers than is conventionally available using casts or splints, thereby decreasing the likelihood of joint stiffness.

A further object resides in providing a low profile for a fracture brace worn on the hand which will allow the wearer or patient to engage in active lifestyles and it is particularly suited to be worn in sporting and industrial settings.

### BRIEF DESCRIPTION OF THE DRAWINGS

The features of the present invention which are believed to be novel are set forth with particularity in the appended claims. The present invention, both as to its organization and manner of operation, together with further objects and advantages thereof, may best be understood with reference to the following description, taken in connection with the accompanying drawings in which:

FIG. 1 a prospective view of the novel low profile metacarpal fracture brace incorporating the present invention illustrating the strap assembly laid out for preparatory for placing the brace on the fractured hand of the user;

FIG. 2 is a reduced front elevational view illustrating the fracture brace held in position on the hand of the user by the strap assembly;

FIG. 3 is a view similar to the view in FIG. 2 illustrating the back of the fracture brace when worn by the user;

FIG. 4 is an enlarged transverse cross-sectional view of the fracture brace shown in FIG. 3 as taken in the direction of arrows 4-4 thereof; and

FIG. 5 is an enlarged view of the fracture brace shown in FIG. 1 illustrated in the direction of arrows 5-5 thereof.

### BRIEF DESCRIPTION OF PREFERRED EMBODIMENT

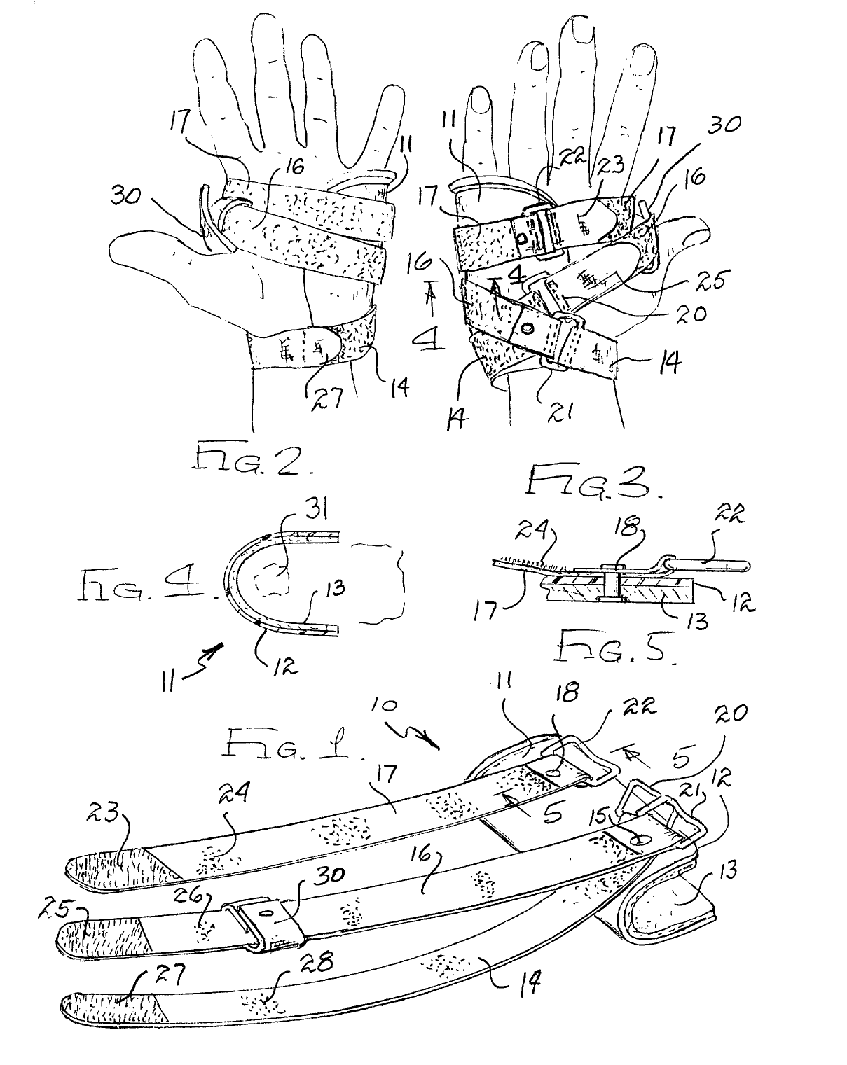

Referring to FIG. 1, the novel low profile metacarpal brace as illustrated in the general direction of arrow 10 which includes a rigid brace member 11 formed with a U-shaped cross section and which includes an outer rigid layer of plastic or plastic-like material and an inner layer of soft, cushioned material. The outer rigid layer is indicated by numeral 12 while the soft inner layer is indicated by numeral 13. An opening extending along the length of the member 11 is used for inserting the fractured hand of the user, preparatory for wrapping the hand and brace member with the strap assembly.

The strap assembly includes multiple straps, wherein one end of each strap is secured to the member 11. For example, a wrist strap 14 as one end selected for attachment to the brace member 11 by means of a rivet 15 that is used in order to retain one end in common for a mid or palm strap 16. An end of an upper palm strap 17 is attached to the member 11 by a separate rivet 18. It is to be particularity noted that each of the respective ends which are secured to the member 11 are also formed in a fold which captures attachment loops. For example, attachment loop 20 is carried at the end strap 14 while loop 21 is carried at the end of strap 16. Loop 22 is carried on the end of strap 17. The loops are intended to insertably receive the free ends of the respective straps after the straps have been wrapped around the member 11 and the injured or fractured hand of the user. For example, end 23 of the upper or top strap 17 is provided with one component of a two-component fastener and is identified by numeral 23 and is intended to be inserted through the loop 22 and folded over the rung of the loop for attachment to a second component of the two-component fastener which is carried on the outer surface of strap 17 and is indicated by numeral 24. The two-component fastener is of a hook and pile type so that an adjustable fit can readily be achieved without having to align fastener components, such as is required by conventional buckles or the like. The fastener component 23 may be of a hook construction while the pile construction 24 is placed along the entire length of the strap from the component 23 to the fastener 18. This construction is illustrated more clearly in FIG. 5. It is to be understood that straps 14 and 16 are constructed in the same manner as described with respect to strap 17 and fasten in a similar manner in that the free end of strap 16 carrying a first component 25 is passed through loop 20 and folded over for attachment with the second component of the hook and pile fastener as indicated by numeral 26. With respect to strap 14, the free end carrying the first fastening component 27 is passed through loop 21 and folded over the rung of the loop for attachment to the second component 28 carried on the outside of strap 14.

Each of the respective straps 14, 16 and 17 are composed of a cloth or fabric material with the first and second components of the two-component fastener carried on the outside surfaces of the strap material. The straps are flexible and are soft so as not to cause discomfort when the straps are wound about the brace member 11 and the hand of the user.

The mid-palm strap 16 carries a spacer cushion member 30 which may be detachably connected to the strap 16 and may be slid along the mid-section of the strap to a desired location. It is intended that member 30 be placed between the thumb and index finger of the user when the strap assembly has been fastened.

It is noted in FIGS. 2 and 3 that the wrist strap 14 is trained from one rung of the loop 20 around the lower palm and a portion of the wrist of the user so that its free end and component 27 can be passed through the rung of loop 21 and folded over upon itself for securement with the second fastener component 28. In a similar fashion, the mid or palm strap 16 is trained from one rung of the loop 21 and is fastening rivet 15 around the back of the member 11 and across the palm, as shown in FIG. 2, and having its terminating end with the first fastening component 25 inserted through and folded over the exposed rung of loop 20 so that it can be connected with the second component 26 of the fastening means. In a similar fashion, the upper strap 17 progresses from loop 22 across the palm of the user's hand immediately below the fingers as shown in FIG. 2 so that its terminating end with fastener 23 can be inserted through the loop 22 and folded over upon itself for connection to the second component 24 carried on the outside of the strap 17. When the strap assembly is fully connected, the brace member 11 is held in position on the hand of the user and the fingers are totally exposed and permit limited movement.

Referring now to FIG. 4, it can be seen that the member 11 includes a rigid outside layer 12 having an inner soft or cushioned layer 13. When carried on the hand of the user, the rigid member is particularly useful in immobilizing the fifth metacarpal which is indicated by numeral 31.

FIG. 5 illustrates the strap 17 as having the second component 24 covering the entire outside length of the strap which terminates in the free end carrying the first component 23. Also, it can be seen that the strap is held on to the brace member 11 by means of rivet 18 after the end of the strap has been folded through one rung of the loop 22. A similar connection and construction is provided for straps 14 and 16 respectively.

In view of the foregoing, it can be seen that the present invention provides a low profile metacarpal fracture brace which is designed to immobilize fractures of the fourth metacarpal and particularly the fifth metacarpal. The brace and strap assembly wraps around the ulnar border of the hand to approximately the middle of the hand on both palmar and dorsal surfaces. The brace extends from the base of the hypothenar eminence to beyond the 5th metacarpal-phalangeal joint to support the base of the proximal phalanx of the fifth finger. Brace position is maintained by the strap assembly running transversely around the hand. The brace may be manufactured in a variety of sizes as an ambidextrous construction allowing the brace to be worn on either hand. The brace can also be made specifically right or left sided wearing. The brace member material is a heat-molded plastic, such that, if desired, it can adjusted for comfort with a heat gun to soften or contour the material. The brace can be lined with either a soft gel-type padding or can be worn over a cloth stockinette material.

The profile metacarpal fracture brace is much easier and convenient for a physician to apply than other traditional methods of treating fourth and fifth metacarpal fractures such as casts or splints. The compact design allows more freedom of motion of the fingers than those other techniques thereby decreasing the likelihood of joint stiffness. The low-profile allows the brace to be used in active lifestyles and is particularly suited to be worn in sporting and industrial settings. Furthermore, the fracture brace is readily removable by the patient for bathing purposes.

While particular embodiments of the present invention have been shown and described, it will be obvious to those skilled in the art that changes and modifications may be made without departing from this invention in its broader aspects and, therefore, the aim in the appended claims is to cover all such changes and modifications as fall within the true spirit and scope of this invention.

What is claimed is:

1. A low profile metacarpal fracture brace comprising:
a U-shaped member having a rigid outer layer defining an open-ended lateral cavity for insertably receiving and supporting the user's hand adjacent to the metacarpal grouping of hand bones;
a plurality of straps having one end of each strap secured to said member with an opposite end of each strap having a free end;
a buckling system carried on each of said ends of said straps secured to said member for insertably receiving said free ends; and
a releasable securement fastener carried on said straps for securing said member to the hand of the user.

2. The fracture brace defined in claim 1 wherein:
said plurality of straps include a lower wrist strap, a middle palm strap and an upper palm strap;
said member having a front side partially covering the palm of the user's hand and a backside partially covering the back of the user's hand with a curved section joining said front side and said backside so that said U-shaped member is defined;
said one end of each strap fixly secured to said backside; and
said plurality of straps wrap transversely around the user's hand.

3. The fracture brace defined in claim 1 wherein:
said member includes an inner surface and an outer surface; and
a cushion material carried on said inner surface immediately adjacent to the hand of the user.

4. The fracture brace defined in claim 3 wherein:
said member is held by said straps so as to extend from the base of the hypothenar eminence to beyond the fifth metacarpal-phalangeal joint of the user's hand to support the base of the proximal phalanx of the fifth finger.

5. The fracture brace defined in claim 4 wherein:
said securement fastener includes a two-component hook and pile fastener carried on each of said straps respectively.

6. The fracture brace defined in claim 5 wherein:
said one end of each of said straps includes a loop for accommodating insertion of said free end for folding over upon itself to couple said two component hook and pile fastener.

7. A low profile metacarpal fracture brace worn on a hand of a user comprising:
an elongated member of U-shaped cross-section having an internal recess with an upper open opposite end joined by a lower lateral entrance leading into said internal recess;
said member composed of a hard and rigid composition providing a shell having a top section adjacent the back of the user's hand when worn by the user, an under section adjacent the palm of the user's hand when worn by the user and an arcuate section joining said top section with said under section, cooperating to define said recess;
said shell having an outer surface and an inner surface;
a cushion material secured to said shell inner surface;
an elongated strap means having a first end secured to said outer surface of said shell and a second end terminating in a free end with a mid-section integrally connecting said first end with said second end;
said first end of said strap means having at least one loop permitting insertion of said free end therethrough for folding over upon itself allowing said free end to overlap against said mid-section;
a two-component fastener means carried on said free end and said mid-section respectively for releasably connecting said strap means; and
said strap means being wrapped transversely about said shell and the hand of the user when said member is worn by said user.

8. The fracture brace defined in claim 7 wherein:
said strap means includes at least three elongated straps;
a first and a second strap of said strap means secured together to said shell immediately adjacent to said lower end thereof constituting a wrist strap and a mid-strap respectively;
a third strap of said strap means secured at its one end to said shell immediately adjacent to said upper end thereof constituting a finger strap;
said mid-strap and said finger strap adapted to wrap about the user's hand across the user's palm and between the user's thumb and index finger with said wrist strap adapted to wrap about the wrist of the user.

9. The fracture brace defined in claim 8 including:
a spaced area of soft composition carried on said midsection of said mid-strap for positioning between the thumb and index finger of the user.

10. The fracture brace defined in claim 9 wherein:
said fastener means includes a hoop and pile fastener on each of said straps of said strap means.

11. The fracture brace defined in claim 10 wherein:
said member insertably receives a portion of the user's hand in said recess whereby said member extends from the base of the hypothenar eminence to beyond the fifth metacarpal-phalangeal joint to support the base of the proximal phalanx of the fifth finger.
